(19) United States
(12) Patent Application Publication
(10) Pub. No.: US 2004/0106643 A1
(43) Pub. Date: Jun. 3, 2004

(54) Tropane derivatives and their use as monoamine neurotransmitter re-uptake inhibitors

(76) Inventors: Alex Haarh Gouliaev, Vekso Sj. (DK); Bjarne H. Dahl, Allerod (DK); Dan Peters, Malmo (SE)

Correspondence Address: BIRCH STEWART KOLASCH & BIRCH, PO BOX 747, FALLS CHURCH, VA, 22040-0747, US

(21) Appl. No.: 10/477,540

(22) PCT Filed: May 23, 2002
(86) PCT No.: PCT/DK02/00346

(51) Int. Cl. A61K 31/46

(52) U.S. Cl. 514/304; 546/124; 546/125

(57) ABSTRACT

This invention relates to tropane derivatives. In other aspects the invention relates to the use of these compounds in a method for therapy and to pharmaceutical compositions comprising the optically active isomer of the invention.

### TECHNICAL FIELD

This invention relates to tropane derivatives.

In other aspects the invention relates to the use of these compounds in a method for therapy and to pharmaceutical compositions comprising the optically active isomer of the invention.

### BACKGROUND ART

EP 604355, EP 604352, U.S. Pat. No. 5,444,070, EP 604354, and WO 97/30997 describe tropane derivatives and their use as mixed monoamine neurotransmitter re-uptake inhibitors. The documents specifically disclose a number of (1R,2R,3S)-2,3-disubstituted tropane derivatives and (1R,2S,3S)-2,3-disubstituted tropane derivatives.

However, there is a continued strong need to find compounds with an optimised biochemical profile as regards the activity on reuptake of the monoamine neurotransmitters serotonin, dopamine and noradrenaline, such as the ratio of the serotonin reuptake versus the noradrenaline and dopamine activity.

Furthermore, there is a strong need to find effective compounds, which structurally and synthetically wise are unrelated to cocaine.

### SUMMARY OF THE INVENTION

Therefore, in its first aspect, the invention provides a disubstituted tropane derivative of general formula Ia or Ib,

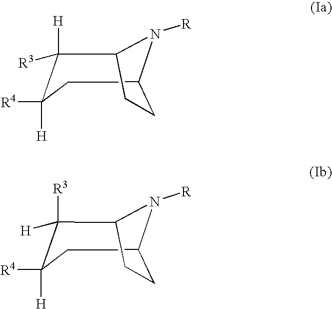

or a pharmaceutically acceptable addition salt thereof or the N-oxide thereof.

In its second aspect the invention provides a pharmaceutical composition comprising a therapeutically effective amount of a compound of the invention together with at least one pharmaceutically-acceptable carrier, excipient or diluent.

In a further aspect the invention provides a method of treatment, prevention or alleviation of a disease or a disorder or a condition of a living animal body, including a human, which disorder, disease or condition is responsive to inhibition of monoamine neurotransmitter re-uptake in the central nervous system (CNS), which method comprises the step of administering to such a living animal body in need thereof a therapeutically effective amount of a compound of the invention.

Other objects of the invention will be apparent to the person skilled in the art from the following detailed description and examples.

### DETAILED DISCLOSURE OF THE INVENTION

In its first aspect, the invention provides a disubstituted tropane derivative of general formula (Ia) or (Ib),

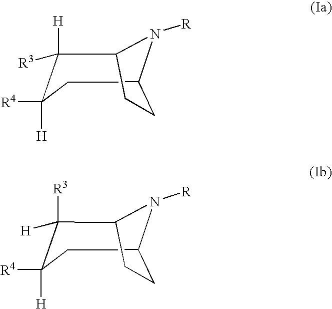

or a pharmaceutically acceptable addition salt thereof or the N-oxide thereof, wherein

R is hydrogen, alkyl, alkenyl, alkynyl, cycloalkyl, cycloalkylalkyl or 2-hydroxyethyl;

R³ is

CH₂—X—R′,

wherein X is O, S, or NR″;

wherein R″ is hydrogen or alkyl;. and

R′ is alkyl, alkenyl, alkynyl, cycloalkyl, cycloalkylalkyl, or —CO-alkyl;

heteroaryl which may be substituted one or more times with

alkyl, cycloalkyl, or cycloalkylalkyl;

phenyl which may be substituted one or more times with substituents selected from the group consisting of halog n, CF₃, CN, alkoxy, alkyl, alkenyl, alkynyl, amino, nitro, and heteroaryl;

phenylphenyl;

pyridyl which may be substituted one or more times with substituents selected from the group consisting of halogen, CF₃, CN, alkoxy, alkyl, alkenyl, alkynyl, amino, nitro, and heteroaryl;

thienyl which may be substituted one or more times with substituents selected from the group consisting of halogen, CF₃, CN, alkoxy, alkyl, alkenyl, alkynyl, amino, nitro, and heteroaryl; or

benzyl which may be substituted one or more times with substituents selected from the group consisting of halogen, CF₃, CN, alkoxy, alkyl, alkenyl, alkynyl, amino, nitro, and heteroaryl; or

(CH₂)ₙCO₂R¹¹, COR¹¹, or CH₂R¹²;

wherein R¹¹ is

alkyl, cycloalkyl, or cycloalkylalkyl;

phenyl which may be substituted one or more times with substituents selected from the group consisting of halogen, CF₃, CN, alkoxy, alkyl, alkenyl, alkynyl, amino, nitro, and heteroaryl;

phenylphenyl;

pyridyl which may be substituted one or more times with substituents selected from the group consisting of halogen, CF₃, CN, alkoxy, alkyl, alkenyl, alkynyl, amino, nitro, and heteroaryl;

thienyl which may be substituted one or more times with substituents selected from the group consisting of halogen, CF₃, CN, alkoxy, alkyl, alkenyl, alkynyl, amino, nitro, and heteroaryl; or

benzyl;

n is 0 or 1; and

R¹² is

O-phenyl which may be substituted one or more times with substituents selected from the group consisting of halogen, CF₃, CN, alkoxy, alkyl, alkenyl, alkynyl, amino, nitro, and heteroaryl; or

O—CO-phenyl which may be substituted one or more times with substituents selected from the group consisting of halogen, CF₃, CN, alkoxy, alkyl, alkenyl, alkynyl, amino, nitro, and heteroaryl;

R⁴ is

3,4-methylenedioxyphenyl or

phenyl, benzyl, naphthyl, or heteroaryl all of which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

In its second aspect, the invention provid s a pharmaceutical composition, comprising a therapeutically effective amount of a compound of the invention, or a pharmaceutically acceptable addition salt thereof, together with at least one pharmaceutically acceptable carrier, excipient or diluent.

In a further aspect, the invention provides the use of a compound of the invention, or a pharmaceutically acceptable addition salt thereof, for the manufacture of a pharmaceutical composition for the treatment, prevention or alleviation of a disease or a disorder or a condition of a mammal, including a human, which disease, disorder or condition is responsive to inhibition of monoamine neurotransmitter re-uptake in the central nervous system.

In a still further aspect, the invention relates to a method for treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound of the invention.

In one embodiment, the invention relates to compounds of formula (Ia). In a second embodiment, the invention relates to compounds of formula (Ib). In a further embodiment, the invention relates to (1S,2S,3R,5R)-8-aza-bicyclo[3.2.1]octane derivatives. In a still further embodiment, the invention relates to (1S,2R,3R,5R)-8-aza-bicyclo [3.2.1]octane derivatives.

In a further embodiment, R³is

1,2,4-oxadiazol-3-yl which may by substituted in the 5 position with

alkyl, cycloalkyl, or cycloalkylalkyl;

phenyl which may be substituted one or more times with substituents selected from the group consisting of halogen, CF₃, CN, alkoxy, alkyl, alkenyl, alkynyl, amino, nitro, and heteroaryl;

phenylphenyl; or

benzyl which may be substituted one or more times with substituents selected from the group consisting of halogen, CF₃, CN, alkoxy, alkyl, alkenyl, alkynyl, amino, nitro, and heteroaryl; or

1,2,4-oxadiazol-5-yl which may by substituted in the 3 position with

alkyl, cycloalkyl, or cycloalkylalkyl;

phenyl which may be substituted one or more times with substituents selected from the group consisting of halogen, CF₃, CN, alkoxy, alkyl, alkenyl, alkynyl, amino, nitro, and heteroaryl;

phenylphenyl;

benzyl which may be substituted one or more times with substituents selected from the group consisting of halogen, CF₃, CN, alkoxy, alkyl, alkenyl, alkynyl, amino, nitro, and heteroaryl;

pyridyl which may be substituted one or more times with substituents selected from the group consisting of halogen , CF₃, CN, alkoxy, alkyl, alkenyl, alkynyl, amino, nitro and heteroaryl; or

thienyl which may be substituted one or more times with substituents selected from the group consisting of halogen, CF₃, CN, alkoxy, alkyl, alkenyl, alkynyl, amino, nitro and heteroaryl.

In still further embodiment, R³ is

CH₂—X—R′,

wherein X is O, S, or NR″;

wherein R″ is hydrogen or alkyl; and

R′ is alkyl, alkenyl, alkynyl, cycloalkyl, cycloalkylalkyl, or —CO-alkyl.

In a further embodiment, R⁴ is

phenyl, which is substituted once or twice with substituents selected from the group consisting of halogen, CF₃, CN, alkoxy, cycloalkoxy, alkyl, cycloalkyl, alkenyl, alkynyl, amino, nitro, and heteroaryl.

In a further embodiment, R is hydrogen, alkyl, alkenyl, alkynyl, cycloalkyl, cycloalkylalkyl, or 2-hydroxyethyl;

R³ is

CH₂—X—R′,

wherein X is O, S, or NR″;

wherein R″ is hydrogen or alkyl; and

R′ is alkyl, alkenyl, alkynyl, cycloalkyl, cycloalkylalkyl, or —CO-alkyl; or

(CH₂)ₙCO₂R¹¹, or COR¹¹;

wherein R¹¹ is alkyl, cycloalkyl, or cycloalkylalkyl; and n is 0 or 1;

R⁴ is

phenyl which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

In a still further embodiment, R is hydrogen or alkyl;

R³ is

CH₂—X—R′,

wherein X is O or S; and

R′ is hydrogen or alkyl; or

(CH₂)ₙCO₂R¹¹;

wherein R¹¹ is alkyl; and n is 0 or 1;

R⁴ is

phenyl which may be substituted once or twice with substitu nts selected from the group consisting of halogen, CF₃, and CN.

In a special embodiment, R is hydrogen. In a further embodiment, R is alkyl, such as methyl.

In a further special embodiment, R³ is CH₂—X—R′, wherein X is O; and R′ is hydrogen or alkyl. In one embodiment, R′ is hydrogen. In a second embodiment, R′ is methyl. In a further embodiment, R′ is ethyl. In a special embodiment, R⁴ is phenyl which may be substituted once or twice with substituents selected from the group consisting of halogen, CF₃, and CN. In one embodiment R⁴ is phenyl which may be substituted once or twice with halogen, such as chlorine. In a second embodiment, R⁴ is phenyl substituted once or twice with chlorine. In a special embodiment, R⁴ is phenyl 3,4-dichlorophenyl.

In a special embodiment, the compound of general formula I is selected from:

(1S,2R,3R,5R)-3-(3,4-Dichlorophenyl)-8-methyl-8-aza-bicyclo[3.2.1]octane-2-carboxylic acid ethyl ester;

(1S,2S,3R,5R)-3-(3,4-Dichlorophenyl)-8-methyl-8-aza-bicyclo[3.2.1]octane-2-carboxylic acid ethyl ester;

(1S,2R,3R,5R)-2-Hydroxymethyl-3-(3,4-dichlorophenyl)-tropane;

(1S,2S,3R,5R)-2-Hydroxymethyl-3-(3,4-dichlorophenyl)-tropane;

(1S,2R,3R,5R)-2-Ethoxymethyl-3-(3,4-dichlorophenyl)-tropane;

(1S,2S,3R,5R)-2-Ethoxymethyl-3-(3,4-dichlorophenyl)-tropane;

(1S,2R,3R,5R)-2-Ethoxymethyl-3-(3,4-dichlorophenyl)-8H-8-aza-bicyclo[3.2.1]octane;

(1S,2S,3R,5R)-2-Ethoxymethyl-3-(3,4-dichlorophenyl)-8H-8-aza-bicyclo[3.2.1]octane;

or a pharmaceutically acceptable addition salt thereof.

Definition of Substituents

In the context of this invention halogen represents a fluorine, a chlorine, a bromine or an iodine atom.

Alkyl means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

Cycloalkyl means cyclic alkyl of three to seven carbon atoms, including but not limited to cyclopropyl, cyclobutyl, cyclopentyl, and cyclohexyl;

Alkenyl means a group of from two to six carbon atoms, including at least one double bond, for example, but not limited to ethenyl, 1,2- or 2,3-propenyl, or 1,2-, 2,3-, or 3,4-butenyl.

Alkynyl means a group of from two to six carbon atoms, including at least one triple bond, for example, but not limited to ethynyl, 1,2-, 2,3-propynyl, or 1,2-, 2,3- or 3,4-butynyl.

Alkoxy is O-alkyl, wherein alkyl is as defined above.

Acyl is —CO-alkyl wherein alkyl is as defined above.

Cycloalkoxy means O-cycloalkyl, wherein cycloalkyl is as defined above.

Cycloalkylalkyl means cycloalkyl as above and alkyl as above, meaning for example, cyclopropylmethyl.

Amino is NH₂ or NH-alkyl or N-(alkyl)₂, wherein alkyl is as defined above.

Aryl is a carbocyclic aromatic ring system such as phenyl or naphthyl (1-naphthyl or 2-naphthyl).

Heteroaryl is a 5- or 6-membered heterocyclic monocyclic group, for example, but not limited to, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1,2,5-thiadiazol4-yl, 2-imidazolyl, 4-imidazolyl, 5-imidazolyl, 2-pyrrolyl, 3-pyrrolyl, 2-furanyl, 3-furanyl, 2-thienyl, 3-thienyl, 2-pyridyl, 3-pyridyl, 4-pyridyl.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

The compounds of the invention may be prepared in numerous ways. The compounds of the invention and their pharmaceutically acceptable derivatives may thus be prepared by any method known in the art for the preparation of compounds of analogous structure, and as shown in the representative examples which follow.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, th aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

The chemical substance according to the invention may be administered as such or in the form of a suitable prodrug.

The term “prodrug” denotes a compound, which is a drug precursor and which, following administration and absorption, release the drug in vivo via some metabolic process.

Particularly favoured prodrugs are those that increase the bioavailability of the compounds of the invention (e.g. by allowing an orally administrered compound to be more readily-absorbed into the blood) or which enhance delivery of the parent compound to a specific biological compartment (e.g. the brain or lymphatic system).

Thus examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are est rs or amides.

Labelled Compounds

The compounds of the invention may be used in their labelled or unlabelled form. In the context of this invention “label” stands for the binding of a marker to the compound of interest that will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

Methods of Preparation

The compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods, such as those described in EP 604355, EP 604352, U.S. Pat. No. 5,444,070, EP 604354, and WO 97/30997.

The end product of the reaction described herein may be isolated by conventional techniques, e.g. by extraction, crystallisation, distillation, chromatography, etc.

Biological Activity

The compounds of the invention may be tested for its ability to inhibit the reuptake of the monoamine neurotransmitters in synaptosomes, eg such as described in WO 97/30997. Based on the balanced activity observed in these tests the compound of the invention is considered useful for combating diseases, disorders or conditions associated with the dopaminergic, noradrenalinergic and/or serotonergic neural system.

The diseases, disorders or conditions contemplated in this context are eating disorders, obesity, anorexia nervosa, disorders of sleep, panic disorders, social phobia, dementia, senile dementia, pre-senile dementia, memory deficits, memory loss, Alzheimer's disease, chronic fatigue syndrome, anxiety, pseudodementia, Ganser's syndrome, narcolepsy, drug addiction or misuse including cocaine abuse, alcoholism, tobacco abuse, panic disorder, post-traumatic syndrome, migraine, pain, attention deficit hyperactivity disorder, autism, mutism, trichotillomania, Parkinson's disease, depression, attention, alertness, arousal, vigilance, premature ejaculation, and erectile dysfunction.

The compounds of the invention are considered particularly useful for the treatment, prevention or alleviation of depression, pseudodementia, Ganser's syndrome, obsessive compulsive disorders, panic disorders, memory deficits, attention deficit hyperactivity disorder, obesity, anxiety, and eating disorders.

Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefor, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in gen ral also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v. and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

In another aspect the invention provides a method for the treatment, prevention or alleviation of a disease or a disorder or a condition of a living animal body, including a human, which disease, disorder or condition is responsive to inhibition of monoamine neurotransmitter re-uptake in the central nervous system (CNS), and which method comprises administering to such a living animal body, including a human, in need thereof an effective amount of the optically active isomer of the invention.

In a more preferred embodiment the invention provides a method of combating depression, pseudodementia, Ganser's syndrome, obsessive compulsive disorders, panic disorders, memory deficits, attention deficit hyperactivity disorder, obesity, anxiety and eating disorders.

It is at present contemplated that suitable dosage ranges are 0.1 to 1000 milligrams daily, 10-500 milligrams daily, and especially .30-100 milligrams daily, dependent as usual upon the exact mode of administration, form in which administered, the indication toward which the administration is directed, the subject involved and the body weight of the subject involved, and further the preference and experience of the physician or veterinarian in charge. A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

Any possible combination of two or more of the embodiments described herein is comprised within the scope of the present invention.

### EXAMPLES

The invention is further illustrated with reference to the following examples, which are not intended to be in any way limiting to the scope of the invention as claimed.

### Example 1

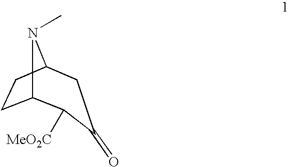

(−)-2-Carbomethoxytropinone (1): Was prepared by a known procedure (J. F. Casale, Forensic Science International, 33 (1987) 275-298).

### Example 2

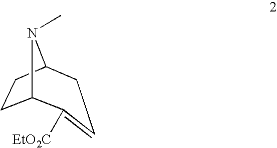

(+)-Ecgonine ethylester (2): To a stirred solution of (−)-2-carbomethoxytropinone (37.4 g, 0.19 mol) in methanol (1.5 l) at −45° C., was added sodium borohydride (37.0 g, 0.98 mol) in small portions, such that the internal temperature was kept between −45° C. and −35° C. The reaction mixture was stirred at −45° C. for 2 hours, and quenched by dropwise addition of concentrated hydrochloric acid (120 ml), while keeping the temperature at −45° C. The reaction mixture was allowed to warm to room temperature and stirred overnight. The reaction mixture was concentrated to a volume of approximately 120 ml, water (500 ml) was added and washed with diethyl ether (3×100 ml). To the aqueous phase was added aqueous ammonia (25%), until basic reaction, and extracted with dichloromethane (4×200 ml). The combined organic phases were dried with sodium sulphate and evaporated to an oil. The oil was dissolved in ethyl acetate (370 ml) and a solution of sodium ethoxide in ethanol (300 ml, 1 M, prepared from sodium (7.0 g, 303 mmol), was added. The resulting solution was heated at reflux for 3 hours, cooled to room-temperature and evaporated to an oil. The residue was solved in toluene (0.5 l) and evaporated to an oil, this was repeated. The product 30 g (79%) was obtained as an oil.

### Example 3

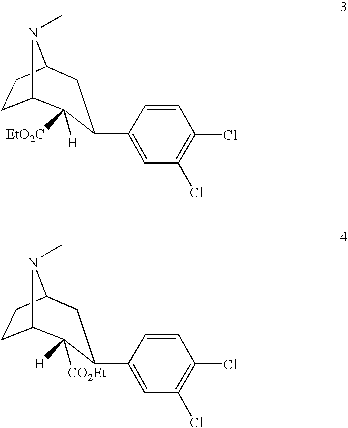

(1S,2R,3R,5R)-3-(3,4-Dichlorophenyl)-8-methyl-8-aza-bicyclo[3.2.1]octane-2-carboxylic acid ethyl ester (3) and (1S,2S,3R,5R)-3-(3,4-dichlorophenyl) -8-methyl-8-aza-bicyclo[3.2.1]octane-2-carboxylic acid ethyl ester (4): A stirred suspension of magnesium turnings (1.25 g, 52 mmol)) in diethyl ether was added bromo-3,4-dichlorobenzene (11.5 g, 49 mmol). The mixture was heated at reflux for 30 minutes and then cooled to −20° C. A solution of (+)-ecgonine ethylester (5.0 g, 25.6 mmol) in toluene (30 ml) was slowly added, while keeping the internal temperature between −15° C. and −10° C. The reaction mixture was stirred at −15° C. for 1½ hour and then added trifluoroacetic acid (8.0 ml) and water (100 ml). Concentrated hydrochloric acid was added until pH=1. The phases were separated and the aqueous phase was washed with diethyl ether (2×100 ml). The aqueous phase was added 25% aqueous ammonia until pH=11 and extracted with dichloromethane (2×100 ml). The combined organic phases were dried using magnesium sulphate and then evaporated to an oil. Yield 7.5 g (86%) of (3) and (4). The isomers (3) and (4) were separated using column chromatography and petroleum ether, diethyl ether, triethylamine (1:1:2%) to give 3.5 g (40%) of (3), Mp 67.5-68.5° C. and 1.7 g (20%) of (4).

### Example 4

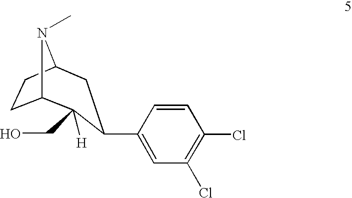

Method A

(1S,2R,3R,5R)-2-Hydroxymethyl-3-(3,4-dichlorophenyl)-tropane (5): A stirred solution of (1S,2R,3R,5R)-3-(3,4-dichlorophenyl)-8-methyl-8-aza-bicyclo[3.2.1]octane-2-carboxylic acid ethyl ester (3), (6.8 g, 20 mmol) in tetrahydrofuran (100 ml) at −40° C. was added lithium aluminum hydride (LiAlH₄) (1.0 g, 26 mmol), while keeping the internal temperature between −40-(−30) ° C. The reaction mixture was left with stirring at −40° C. for 1 h, then quenched by addition of water (10 ml) followed by aqueous sodium hydroxide (10 ml, 4 M). The mixture was filtered and evaporated to an oil. The oil was dissolved in dichloromethane and dried with magnesium sulphate and evaporated to a solid. Yield 5.3 g (88%), Mp. 81-86° C.

### Example 5

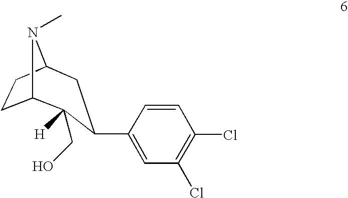

(1S,2S,3R,5R)-2-Hydroxymethyl-3-(3,4-dichlorophenyl)-tropane (6): (1S,2S,3R,5R)-3-(3,4-dichlorophenyl)-8-methyl-8-aza-bicyclo[3.2.1]octane -2-carboxylic acid ethyl ester (4) ( 5.6 g, 17.1 mmol) was reduced according to method A giving 4.4 g (86%) of the title compound.

### Example 6

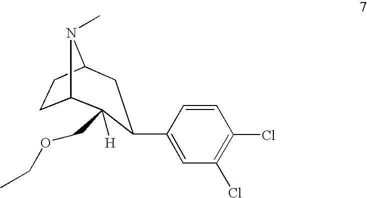

Method B

(1S,2R,3R,5R)-2-Ethoxymethyl-3-(3,4-dichlorophenyl)-tropane (7): (1S,2R,3R,5R) -2-Hydroxymethyl-3-(3,4-dichlorophenyl)-tropane (5 ) (1.0 g) in dry tetrahydrofuran (15 ml) was added sodium hydride (0.25 g, 6.25 mmol) and stirred for 15 minutes. Diethyl sulfate (0.54 ml, 4.1 mmol) was added and the mixture stirred at 50° C. for 3 h. The reaction mixture was poured into water and extracted twice, using diethyl ether (2×50 ml). The organic phases were dried by magnesium sulphate and evaporated to dryness. Column chromatography, using a mixture of dichloromethane, methanol and ammonia (9:1:1%) yielded 200 mg (18%) of the title compound as the free base. Mp. 52.5-54.5° C.

### Example 7

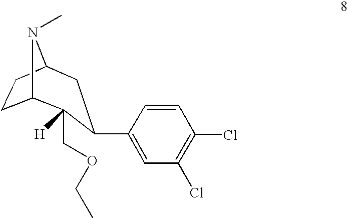

(1S,2S,3R, 5R)-2-Ethoxymethyl-3-(3,4dichlorophenyl)-tropane citrate (8): Was synthesized from (1S,2S,3R,5R)-2-hydroxymethyl-3-(3,4-dichlorophenyl)-tropane (6) (1.5 g) according to method B. Yield 290 mg as the free base. The free base was dissolved in ethanol (10 ml, 96%) and added citric acid (190 mg, 1.0 mmol). The suspension was heated to obtain a clear solution and left for precipitation. The precipitate was isolated by filtration to yield 290 mg (11%) of the title compound. Mp. 161.9-162.3° C.

### Example 8

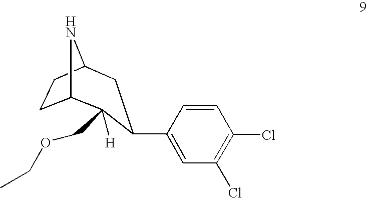

(1S,2R,3R,5R)-2-Ethoxymethyl-3-(3,4-dichlorophenyl)-8H-8-aza-bicyclo [3.2.1]octane fumarate (9): To a mixture of (1S,2R,3R,5R)-2-ethoxymethyl-3-(3,4-dichlorophenyl)-tropane (7) (0.5 g, 1.53 mmol) and toluene (5 ml), was added 1-chloroethyl chloroformate (0.25 ml, 2.3 mmol). The mixture was stirred stirred at 100° C. for 48 hours. Water (30 ml) was added and the reaction mixture stirred for 6 hours at 75° C. The mixture was extracted with diethyl ether (2×50 ml). The organic phase were dried with magnesium sulphate and evaporated. Column chromatography, using dichloromethane, methanol and ammonia (9, 1, 1%). Yield 0.33 g (69%). A solution of fumaric acid (4.1 ml, 0.078 M) in diethyl ether and MeOH (9:1) was added to the free base (100 mg, 0.32 mmol). The mixture was stirred for one hour. The precipitate was isolated by filtration yielding 110 mg (0.25 mmol) of the title compound. Mp 145-147.5° C.

### Example 9

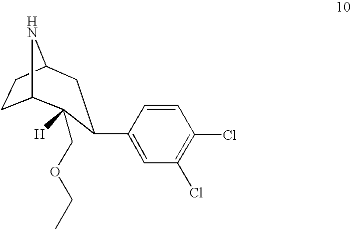

(1S,2S,3R,5R)-2-Ethoxymethyl-3-(3,4-dichlorophenyl)-8H-8-aza-bicyclo [3.2.1]octane fumarate (10): The compound was synthesized from (1S,2S,3R,5R)-2-ethoxymethyl-3-(3,4-dichlorophenyl)-tropane (8) according to method B. Mp 219-220° C.

### Example 10

The following compounds and pharmaceutically acceptable salts thereof is prepared analogously using the methods as described above and in EP 604355, EP 604352, U.S. Pat. No. 5,444,070, EP 604354, and WO 97/30997.

(1S,2S,3R,5R)-2-(3-Cyclopropyl-1,2,4-oxadiazol-5-yl)-3-(4-fluorophenyl)tropane;

(1S,2S,3R,5R)-2-(3-Phenyl-1,2,4-oxadiazol-5-yl)-3-(4-fluorophenyl)tropane;

(1S,2S,3R,5R)-2-(3-Phenyl-1,2,4-oxadiazol-5-yl)-3-(4-methylphenyl)-tropane;

(1S,2S,3R,5R)-2-(3-Benzyl-1,2,4-oxadiazol-5-yl)-3-(4-fluorophenyl)tropane;

(1S,2S,3R,5R)-2-(3-(4-Phenyl-phenyl)-1,2,4-oxadiazol-5-yl)-3-(4-fluorophenyl)tropane;

(1S,2S,3R,5R)-2-(3-Phenyl-1,2,4-oxadiazol-5-yl)-3-(2-naphthyl)tropane;

(1S,2R,3R,5R)-2-(3-Cyclopropyl-1,2,4-oxadiazol-5-yl)-3-(4-fluorophenyl)tropane;

(1S,2R,3R,5R)-2-(3-Phenyl-1,2,4-oxadiazol-5-yl)-3-(4-fluorophenyl)tropane;

(1S,2R,3R,5R)-2-(3-Phenyl-1,2,4-oxadiazol-5-yl)-3-(4-methylphenyl)-tropane;

(1S,2R,3R,5R)-2-(3-Benzyl-1,2,4-oxadiazol-5-yl)-3-(4-fluorophenyl)tropane;

(1S,2R,3R,5R)-2-(3-(4-Phenyl-phenyl)-1,2,4-oxadiazol-5-yl)-3-(4-fluorophenyl)tropane;

(1S,2R,3R,5R)-2-(3-Phenyl-1,2,4-oxadiazol-5-yl)-3-(2-naphthyl)tropane;

(1S,2S,3R,5R)-2-Methoxymethyl-3-(3,4-dichlorophenyl)-tropane;

(1S,2S,3R,5R)-2-Isopropoxymethyl-3-(3,4-dichlorophenyl)-tropane;

(1S,2S,3R,5R)-2-Cyclopropylmethyloxymethyl-3-(3,4-dichlorophenyl)-tropane;

(1S,2S,3R,5R)-2-Methoxymethyl-3-(4-chlorophenyl)-tropane;

(1S,2S,3R,5R)-N-Normethyl-2-methoxymethyl-3-(4-chlorophenyl)-tropane;

(1S,2S,3R,5R)-2-Ethoxymethyl-3-(4-chlorophenyl)-tropane;

(1S,2S,3R,5R)-N-Normethyl-2-methoxymethyl-3-(3,4-dichlorophenyl)-tropane;

(1S,2S,3R,5R)-N-Normethyl-2-ethoxymethyl-3-(4-chlorophenyl)-tropane;

(1S,2S,3R,5R)-N-Normethyl-2-cyclopropylmethyloxymethyl-3-(4-chlorophenyl)-tropane;

(1S,2S,3R,5R)-2-Cyclopropylmethyloxymethyl-3-(4-chlorophenyl)-tropane;

(1S,2S,3R,5R)-2-Ethylthiomethyl-3-(3,4-dichlorophenyl)-tropane;

(1S,2S,3R,5R)-2-Hydroxymethyl-3-(4-fluorophenyl)tropane;

(1S,2S,3R,5R)-2-Hydroxymethyl-3-(3,4-dichlorophenyl)tropane;

(1S,2S,3R,5R)-N-Normethyl-N-(tert-butoxycarbonyl)-2-hydroxymethyl -3-(3,4-dichlorophenyl)tropane;

(1S,2S,3R,5R)-2-Hydroxymethyl-3-(4-chlorophenyl)tropane;

(1S,2R,3R,5R)-2-Methoxymethyl-3-(3,4-dichlorophenyl)-tropane;

(1S,2R,3R,5R)-2-Isopropoxymethyl-3-(3,4-dichlorophenyl)-tropane;

(1S,2R,3R,5R)-2-Cyclopropylmethyloxymethyl-3-(3,4-dichlorophenyl)-tropane;

(1S,2R,3R,5R)-2-Methoxymethyl-3-(4-chlorophenyl)-tropane;

(1S,2R,3R,5R)-N-Normethyl-2-methoxymethyl-3-(4-chlorophenyl)-tropane;

(1S,2R,3R,5R)-2-Ethoxymethyl-3-(4-chlorophenyl)-tropane;

(1S,2R,3R,5R)-N-Normethyl-2-methoxymethyl-3-(3,4-dichlorophenyl)-tropane;

(1S,2R,3R,5R)-N-Normethyl-2-ethoxymethyl-3-(4-chlorophenyl)-tropane;

(1S,2R,3R,5R)-N-Normethyl-2-cyclopropylmethyloxymethyl-3-(4-chlorophenyl)-tropane;

(1S,2R,3R,5R)-2-Cyclopropylmethyloxymethyl-3-(4-chlorophenyl)-tropane;

(1S,2R,3R,5R)-2-Ethylthiomethyl-3-(3,4-dichlorophenyl)-tropane;

(1S,2R,3R,5R)-2-Hydroxymethyl-3-(4-fluorophenyl)tropane;

(1S,2R,3R,5R)-2-Hydroxymethyl-3-(3,4-dichlorophenyl)tropane;

(1S,2R,3R,5R)-N-Normethyl-N-(tert-butoxycarbonyl)-2-hydroxymethyl -3-(3,4-dichlorophenyl)tropane;

(1S,2R,3R,5R)-2-Hydroxymethyl-3-(4-chlorophenyl)tropane;

(1S,2S,3R,5R)-2-(3-(2-Furanyl)-1 ,2,4-oxadiazol-5-yl)-3-(3,4-dichlorophenyl)-tropane;

(1S,2S,3R,5R)-2-(3-(3-Pyridyl)-1,2,4-oxadiazol-5-yl)-3-(3,4-dichlorophenyl)-tropane;

(1S,2S,3R,5R)-N-Normethyl-N-ally-2-(3-(4-pyridyl)-1,2,4-oxadiazol-5-yl) -3-(3,4-dichlorophenyl)-tropane;

(1S,2S,3R,5R)-N-Normethyl-N-ethyl-2-(3-(4-pyridyl)-1,2,4-oxadiazol-5-yl)-3-(3,4-dichlorophenyl)-tropane;

(1S,2S,3R,5R)-N-Normethyl-N-(2-hydroxyethyl)-2-(3-(4-pyridyl)-1,2,4-oxadiazol-5-yl) -3-(3,4-dichlorophenyl)-tropane;

(1S,2S,3R,5R)-N-Normethyl-2-(3-(4-pyridyl)-1,2,4-oxadiazol-5-yl)-3-(3,4-dichlorophenyl)-tropane;

(1S,2S,3R,5R)-N-Normethyl-N-allyl-2-(3-(3-pyridyl)-1,2,4-oxadiazol-5-yl)-3-(3,4-dichlorophenyl)-tropane;

(1S,2S,3R,5R)-N-Normethyl-N-allyl-2-(3-(2-pyridyl)-1,2,4-oxadiazol-5-yl) -3-(3,4-dichlorophenyl)-tropane;

(1S,2S,3R,5R)-2-(3-(2-Thienyl)-1,2,4-oxadiazol-5-yl)-3-(4-chlorophenyl)-tropane;

(1S,2S,3R,5R)-2-(3-(2-Thienyl)-1,2,4-oxadiazol-5-yl)-3-(3,4-dichlorophenyl)-tropane;

(1S,2S,3R,5R)-2-(3-(4-Pyridyl)-1,2,4-oxadiazol-5-yl)-3-(3,4-dichlorophenyl)-tropane;

(1S,2S,3R,5R)-2-(3-(2-Pyridyl)-1,2,4-oxadiazol-5-yl)-3-(3,4-dichlorophenyl)-tropane;

(1S,2S,3R,5R)-2-(3-(4-Pyridyl)-1,2,4-oxadiazol-5-yl)-3-(4-chlorophenyl)-tropane;

(1S,2S,3R,5R)-2-(3-(3-Pyridyl)-1,2,4-oxadiazol-5-yl)-3-(4-chlorophenyl)-tropane;

(1S,2S,3R,5R)-2-(3-(2-Pyridyl)-1,2,4-oxadiazol-5-yl)-3-(4-chlorophenyl)-tropane;

(1S,2R,3R,5R)-2-(3-(2-Furanyl)-1,2,4-oxadiazol-5-yl)-3-(3,4-dichlorophenyl)-tropane;

(1S,2R,3R,5R)-2-(3-(3-Pyridyl)-1,2,4-oxadiazol-5-yl)-3-(3,4-dichlorophenyl)-tropane;

(1S,2R,3R,5R)-N-Normethyl-N-allyl-2-(3-(4-pyridyl)-1,2,4-oxadiazol-5-yl) -3-(3,4-dichlorophenyl)-tropane;

(1S,2R,3R,5R)-N-Normethyl-N-ethyl-2-(3-(4-pyridyl)-1,2,4-oxadiazol-5-yl) -3-(3,4-dichlorophenyl)-tropane;

(1S,2R,3R,5R)-N-Normethyl-N-(2-hydroxyethyl) -2-(3-(4-pyridyl)-1,2,4-oxadiazol-5-yl) -3-(3,4-dichlorophenyl)-tropan

(1S,2R,3R,5R)-N-Normethyl-2-(3-(4-pyridyl)-1,2,4-oxadiazol-5-yl) -3-(3,4-dichlorophenyl)-tropane;

(1S,2R,3R,5R)-N-Normethyl-N-allyl-2-(3-(3-pyridyl)-1,2,4-oxadiazol-5-yl) -3-(3,4-dichlorophenyl)-tropane;

(1S,2R,3R,5R)-N-Normethyl-N-allyl-2-(3-(2-pyridyl)-1,2,4-oxadiazol-5-yl) -3-(3,4-dichlorophenyl)-tropane;

(1S,2R,3R,5R)-2-(3-(2-Thienyl)-1,2,4-oxadiazol-5-yl)-3-(4-chlorophenyl)-tropane;

(1S,2R,3R,5R)-2-(3-(2-Thienyl)-1,2,4-oxadiazol-5-yl)-3-(3,4-dichlorophenyl)-tropane;

(1S,2R,3R,5R)-2-(3-(4-Pyridyl)-1,2,4-oxadiazol-5-yl)-3-(3,4-dichlorophenyl)-tropane;

(1S,2R,3R,5R)-2-(3-(2-Pyridyl)-1,2,4-oxadiazol-5-yl)-3-(3,4-dichlorophenyl)-tropane;

(1S,2R,3R,5R)-2-(3-(4-Pyridyl)-1,2,4-oxadiazol-5-yl)-3-(4-chlorophenyl)-tropane;

(1S,2R,3R,5R)-2-(3-(3-Pyridyl)-1,2,4-oxadiazol-5-yl)-3-(4-chlorophenyl)-tropane;

(1S,2R,3R,5R)-2-(3-(2-Pyridyl)-1,2,4-oxadiazol-5-yl)-3-(4-chlorophenyl)-tropane;

(1S,2S,3R,5R)-2-(3-Phenyl-1,2,4-oxadiazol-5-yl)-3-(4-fluorophenyl)-tropane;

(1S,2S,3R,5R)-2-(3-Phenyl-1,2,4-oxadiazol-5-yl)-3-(4-methylphenyl)-tropane;

(1S,2S,3R,5R)-2-(3-Benzyl-1,2,4-oxadiazol-5-yl)-3-(4-fluorophenyl)-tropane;

(1S,2S,3R,5R)-2-(3-(4-Phenylphenyl)-1,2,4-oxadiazol-5-yl)-3-(4-fluorophenyl)-tropane;

(1S,2S,3R,5R)-2-(3-Phenyl-1,2,4-oxadiazol-5-yl)-3-(2-naphthyl)-tropane;

(1S,2R,3R,5R)-2-(3-Phenyl-1,2,4-oxadiazol-5-yl)-3-(4-fluorophenyl)-tropane;

(1S,2R,3R,5R)-2-(3-Phenyl-1,2,4-oxadiazol-5-yl)-3-(4-methylphenyl)-tropane;

(1S,2R,3R,5R)-2-(3-Benzyl-1,2,4-oxadiazol-5-yl)-3-(4-fluorophenyl)-tropane;

(1S,2R,3R,5R)-2-(3-(4-Phenylphenyl)-1,2,4-oxadiazol-5-yl)-3-(4-fluorophenyl)-tropane;

(1S,2R,3R,5R)-2-(3-Phenyl-1,2,4-oxadiazol-5-yl)-3-(2-naphthyl)-tropane;

(1S,2S,3R,5R)-2-(4-Chlorophenoxy-methyl)-3-(4-fluorophenyl)-tropane;

(1S,2S,3R,5R)-2-(4-Chlorophenoxy-methyl)-3-(4-fluorophenyl)-tropane;

(1S,2S,3R,5R)-2-(4-Chlorophenoxy-methyl)-3-(3,4-dichlorophenyl)-tropane;

(1S,2S,3R,5R)-2-(4-Chlorophenoxy-methyl)-3-(4-methylphenyl)-tropane;

(1S,2S,3R,5R)-2-(4-Benzoyloxy-methyl)-3-(4fluorophenyl)-tropane;

(1S,2S,3R,5R)-2-Carbomethoxy-3-(2-naphthyl)-tropane;

(1S,2S,3R,5R)-2-Carbomethoxy-3-(3,4-dichlorophenyl)-tropane;

(1S,2S,3R,5R)-2-Carbomethoxy-3-benzyl-tropane;

(1S,2S,3R,5R)-2-Carbomethoxy-3-(4-chlorophenyl)-tropane;

(1S,2S,3R,5R)-2-Carbomethoxy-3-(4-methylphenyl)-tropane;

(1S,2S,3R,5R)-2-Carbomethoxy-3-(1-naphthyl)-tropane;

(1S,2S,3R,5R)-2-Carbomethoxy-3-(4-phenylphenyl)-tropane;

(1S,2S,3R,5R)-2-Carbomethoxy-3-(4-t-butyl-phenyl)-tropane;

(1S,2S,3R,5R)-2-(4-Fluoro-benzoyl)-3-(4-fluorophenyl)-tropane

(1S,2R,3R,5R)-2-(4-Chlorophenoxy-methyl)-3-(4-fluorophenyl)-tropane;

(1S,2R,3R,5R)-2-(4-Chlorophenoxy-methyl)-3-(4-fluorophenyl)-tropane;

(1S,2R,3R,5R)-2-(4-Chlorophenoxy-methyl)-3-(3,4-dichloroph nyl)-tropane;

(1S,2R,3R,5R)-2-(4-Chlorophenoxy-methyl)-3-(4-methylphenyl)-tropane;

(1S,2R,3R,5R)-2-(4-Benzoyloxy-methyl)-3-(4-fluorophenyl)-tropane;

(1S,2R,3R,5R)-2-Carbomethoxy-3-(2-naphthyl)-tropane;

(1S,2R,3R,5R)-2-Carbomethoxy-3-(3,4-dichlorophenyl)-tropane;

(1S,2R,3R,5R)-2-Carbomethoxy-3-benzyl-tropane;

(1S,2R,3R,5R)-2-Carbomethoxy-3-(4-chlorophenyl)-tropane;

(1S,2R,3R,5R)-2-Carbomethoxy-3-(4-methylphenyl)-tropane;

(1S,2R,3R,5R)-2-Carbomethoxy-3-(1-naphthyl)-tropane;

(1S,2R,3R,5R)-2-Carbomethoxy-3-(4-phenylphenyl)-tropane;

(1S,2R,3R,5R)-2-Carbomethoxy-3-(4-t-butyl-phenyl)-tropane;

(1S,2R,3R,5R)-2-(4-Fluoro-benzoyl)-3-(4-fluorophenyl)-tropane.

1. A disubstituted tropane derivative of general formula (Ia) or (Ib), 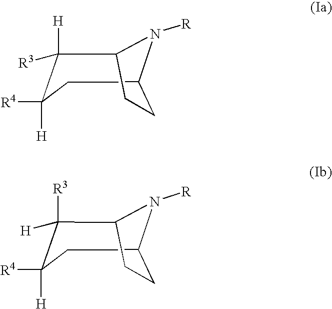
or a pharmaceutically acceptable addition salt thereof or the N-oxide thereof, wherein
R is hydrogen, alkyl, alkenyl, alkynyl, cycloalkyl, cycloalkylalkyl, or 2-hydroxyethyl;
R³ is
CH₂—X—R′,
wherein X is O, S, or NR″;
wherein R″ is hydrogen or alkyl; and
R′ is alkyl, alkenyl, alkynyl, cycloalkyl, cycloalkylalkyl, or —CO-alkyl;
heteroaryl which may be substituted one or more times with
alkyl, cycloalkyl, or cycloalkylalkyl;
phenyl which may be substituted one or more times with substituents selected from the group consisting of halogen, CF₃, CN, alkoxy, alkyl, alkenyl, alkynyl, amino, nitro, and heteroaryl;
phenylphenyl;
pyridyl which may be substituted one or more times with substituents selected from the group consisting of halogen, CF₃, CN, alkoxy, alkyl, alkenyl, alkynyl, amino, nitro, and heteroaryl;
thienyl which may be substituted one or more times with substituents selected from the group consisting of halogen, CF₃, CN, alkoxy, alkyl, alkenyl, alkynyl, amino, nitro, and heteroaryl; or
benzyl which may be substituted one or more times with substituents selected from the group consisting of halogen, CF₃, CN, alkoxy, alkyl, alkenyl, alkynyl, amino, nitro, and heteroaryl; or
(CH₂)ₙCO₂R¹¹, COR¹¹, or CH₂R¹²;
wherein R¹¹ is
alkyl, cycloalkyl, or cycloalkylalkyl;
phenyl which may be substituted one or more times with substituents selected from the group consisting of halogen, CF₃, CN, alkoxy, alkyl, alkenyl, alkynyl, amino, nitro, and heteroaryl;
phenylphenyl;
pyridyl which may be substituted one or more times with substituents selected from the group consisting of halogen, CF₃, CN, alkoxy, alkyl, alkenyl, alkynyl, amino, nitro, and heteroaryl;
thienyl which may be substituted one or more times with substituents selected from the group consisting of halogen, CF₃, CN, alkoxy, alkyl, alkenyl, alkynyl, amino, nitro, and heteroaryl; or
benzyl;
n is 0 or 1; and
R¹² is
O-phenyl which may be substituted one or more times with substituents selected from the group consisting of halogen, CF₃, CN, alkoxy, alkyl, alkenyl, alkynyl, amino, nitro, and heteroaryl; or
O—CO-phenyl which may be substituted one or more times with substituents selected from the group consisting of halogen, CF₃, CN, alkoxy, alkyl, alkenyl, alkynyl, amino, nitro, and heteroaryl;
R⁴is
3,4-methylenedioxyphenyl or
phenyl, benzyl, naphthyl, or heteroaryl all of which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

2. The compound according to claim 1, wherein
R³ is
1,2,4-oxadiazol-3-yl which may by substituted in the 5 position with
alkyl, cycloalkyl, or cycloalkylalkyl;
phenyl which may be substituted one or more times with substituents selected from the group consisting of halogen, CF₃, CN, alkoxy, alkyl, alkenyl, alkynyl, amino, nitro, and heteroaryl
phenylphenyl; or
benzyl which may be substituted one or more times with substituents selected from the group consisting of halogen, CF₃, CN, alkoxy, alkyl, alkenyl, alkynyl, amino, nitro, and heteroaryl; or
1,2,4-oxadiazol-5-yl which may by substituted in the 3 position with
alkyl, cycloalkyl, or cycloalkylalkyl;
phenyl which may b substituted one or more times with substituents selected from the group consisting of halogen, CF₃, CN, alkoxy, alkyl, alkenyl, alkynyl, amino, nitro, and heteroaryl;
phenylphenyl;
benzyl which may be substituted one or more times with substituents selected from the group consisting of halogen, CF₃, CN, alkoxy, alkyl, alkenyl, alkynyl, amino, nitro, and heteroaryl;
pyridyl which may be substituted one or more times with substituents selected from the group consisting of halogen , CF₃, CN, alkoxy, alkyl, alkenyl, alkynyl, amino, nitro and heteroaryl; or
thienyl which may be substituted one or more times with substituents selected from the group consisting of halogen, CF₃, CN, alkoxy, alkyl, alkenyl, alkynyl, amino, nitro and heteroaryl.

3. The compound according to claim 1, wherein
R³ is
CH₂—X—R′,
wherein X is O, S, or NR″;
wherein R″ is hydrogen or alkyl; and
R′ is alkyl, alkenyl, alkynyl, cycloalkyl, cycloalkylalkyl, or —CO-alkyl.

4. The compound according to any one of the claims 1-3, wherein
R⁴ is
phenyl, which is substituted once or twice with substituents selected from the group consisting of halogen, CF₃, CN, alkoxy, cycloalkoxy, alkyl, cycloalkyl, alkenyl, alkynyl, amino, nitro, and heteroaryl.

5. The compound according to claim 1, wherein
R is hydrogen, alkyl, alkenyl, alkynyl, cycloalkyl, cycloalkylalkyl, or 2-hydroxyethyl;
R³ is
CH₂—X—R′,
wherein X is O, S, or NR″;
wherein R″ is hydrogen or alkyl; and
R′ is alkyl, alkenyl, alkynyl, cycloalkyl, cycloalkylalkyl, or —CO-alkyl; or
(CH₂)ₙCO₂R¹¹ or COR¹¹;
wherein R¹¹ is alkyl, cycloalkyl, or cycloalkylalkyl; and n is 0 or 1;
R⁴ is
ph nyl which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

6. The compound according to claim 1, wherein
R is hydrogen or alkyl;
R³ is
CH₂—X—R′,
wherein X is O or S; and
R′ is hydrogen or alkyl; or
(CH₂)ₙCO₂R¹¹;
wherein R″ is alkyl; and n is 0 or 1;
R⁴ is
phenyl which may be substituted once or twice with substituents selected from the group consisting of halogen, CF₃, and CN.

7. A compound of claim 1 which is
(1S,2R,3R,5R)-3-(3,4-Dichlorophenyl)-8-methyl-8-aza-bicyclo[3.2.1]octane-2-carboxylic acid ethyl ester;
(1S,2S,3R,5R)-3-(3,4-Dichlorophenyl)-8-methyl-8-aza-bicyclo[3.2.1]octane-2-carboxylic acid ethyl ester;
(1S,2R,3R,5R)-2-Hydroxymethyl-3-(3,4-dichlorophenyl)-tropane;
(1S,2S,3R,5R)-2-Hydroxymethyl-3-(3,4-dichlorophenyl)-tropane;
(1S,2R,3R,5R)-2-Ethoxymethyl-3-(3,4-dichlorophenyl)-tropane;
(1S,2S,3R,5R)-2-Ethoxymethyl-3-(3,4-dichlorophenyl)-tropane;
(1S,2R,3R,5R)-2-Ethoxymethyl-3-(3,4-dichlorophenyl)-8H-8-aza-bicyclo[3.2.1]octane;
(1S,2S,3R,5R)-2-Ethoxymethyl-3-(3,4-dichlorophenyl)-8H-8-aza-bicyclo[3.2.1]octane;
or a pharmaceutically acceptable addition salt thereof.

8. A pharmaceutical composition, comprising a therapeutically effective amount of a compound of any one of claims 1-7, or a pharmaceutically acceptable addition salt thereof, together with at least one pharmaceutically acceptable carrier, excipient or diluent.

9. The use of a compound according to any one of claims 1-7, or a pharmaceutically acceptable addition salt thereof, for the manufacture of a pharmaceutical composition for the treatment, prevention or alleviation of a disease or a disorder or a condition of a mammal, including a human, which disease, disorder or condition is responsive to inhibition of monoamine neurotransmitter re-uptake in the central nervous system.

10. The use according to claim 9, wherein the disease, disorder or condition is depression, pseudodementia, Ganser's syndrome, obsessive compulsive disorders, panic disorders, memory deficits, attention deficit hyperactivity disorder, obesity, anxiety and eating disorders.

11. A method for treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound according to any one of the claims 1-7.

12. The method of claim 11, wherein the disease, disorder or condition is depression, pseudodementia, Ganser's syndrome, obsessive compulsive disorders, panic disorders, memory deficits, attention deficit hyperactivity disorder, obesity, anxiety and eating disorders.
